(19) United States
(12) Patent Application Publication
(10) Pub. No.: US 2017/0209040 A1
(43) Pub. Date: Jul. 27, 2017

(54) VISUAL FUNCTIONS ASSESSMENT USING CONTRASTING STROBIC AREAS

(71) Applicant: Allan N Hytowitz, Alpharetta, GA (US)

(72) Inventor: Allan N Hytowitz, Alpharetta, GA (US)

(21) Appl. No.: 14/868,045

(22) Filed: Sep. 28, 2015

(51) Int. Cl. A61B 3/032; A61B 3/06; A61B 3/036; A61B 3/02; A61B 3/09

(52) U.S. Cl. CPC A61B 3/032; A61B 3/022; A61B 3/09; A61B 3/036; A61B 3/066

(57) ABSTRACT

A test to assess visual functions based upon the vision system's neuro-biological basis of vision requiring changes in stimulus. Multiple contrasting visual stimulus areas are varied by visual arc-area, color, contrast, velocity, strobic rate, size and relative sizes to test visual functions.

### BACKGROUND OF THE INVENTION

Visual acuity (“VA”) is clearness of vision defined as the ability to perceive or resolve a linear minimum angle of separation or “Minimum Angle of Resolution” between two stationary lines or points. Historical measures of VA are defined and tested using unmoving or static images generally composed of high contrast, black letters or pictograms on a white background, or the converse, using images of commonly recognized characters, “Optotypes”, such as Snellen letters, Sloan letters and numbers, Landolt C's, Tumbling E's or pictograms designed for illiterates and children. The human eye's neuro-biologic function that produces visual sensation fundamentally requires changes in light stimulus on a given retinal area rather than strictly resolving static differentiation of two points. A classical demonstration of this phenomena has been described by various researchers using an image that was mechanically stabilized, i.e. not moving on the retina, resulting in the image fully fading and no longer visible within a few seconds.¹ ¹ Ditchburn & Ginsborg, 1952; Pritchard, 1961; Pritchard, Heron, & Hebb, 1960; Riggs & Ratliff, 1952; Riggs, Ratliff, Cornsweet, & Cornsweet, 1953.

As another example, Troxler's Fading² ² Troxler, D. (I.P.V.) (1804), “Uber das Verschwinden gegebener Gegenstände innerhalb unseres Gesichtskreises” [On the disappearance of genven objects from our visual field]. In Himly, K.; Schmidt, J. A. Opthalmologische Bibliothek (in German) 2 (2): 1-53. OCLC 491712012
also demonstrates the importance of visual stimuli area movement showing that visual stimuli that do not change position, or move, rapidly no longer are visually perceived. These retinal stabilization experiments demonstrate that the human visual system has evolved to optimally detect changes in stimulation and motion, which is not directly the primary feature tested using historical VA testing methods.

Historical assessment of multiple features of vision function such as visual acuity, contrast sensitivity, color vision, refraction and perception of distance and depth, commonly utilize stationary targets in which a subject is requested to correctly identify figures composed of text, numbers, or pictograms to arrive at a confusion point defining the perceptual threshold. An advanced testing method in which various aspects of vision performance can assessed leveraging the innate features of the vision system's stimulus varying requirement is herein described.

### APPLICATION

A novel assessment of visual function that uses multiple area, contrasting, scalable, geometric, non-literate images with defined strobic contrast or movement or velocity, termed a “Dynamic Optotype”, to stimulate the vision system's neuro-biologic makeup and improve detection precision, qualitatively or quantitatively, of a variety of visual functions. Research has shown that a Dynamic Optotype composed of multiple, contrasting or segmented areas of variable area, size, color, intensity, velocity, and contrast can provide a more precise measure of visual acuity and other visual functions compared to static back and white characters or pictograms.

The Dynamic Optotype's visual arc-areas movement or strobic contrast provides a multi-area, changing stimulus to the retinal photoreceptors and permits a higher precision of various vision function measurements by summing the effects of a plurality of visual areas stimulation. The Dynamic Optotype will be observed by a subject as either having specific strobic contrast or direction or directions of motion, or having the perception of an illusion of no motion based upon the selected Dynamic Optotype features. The subject's perceptual threshold or test endpoint appears binary, “ON or OFF”, rather than when a statistically meaningful number of errors, misidentifications, or confusions of static letters or pictograms that are based upon linear, minimum angle of resolution as in historical methods. The ON or OFF perception may be applied to assessment of multiple visual functions such as visual acuity, dynamic visual acuity, refraction, distance detection, visual size differentiation, motion, strobic contrast, color detection or sensitivity, and contrast sensitivity.

The Dynamic Optotype test uses the detection of strobic contrast or movement of a plurality of calibrated, contrasting areas displayed over significantly larger total area of retinal receptors and are more precise than detection of linear angles of separation between stationary points as with historical visual acuity testing. The perception of the visual angle movement of the stimulus areas may be emulated by the strobic alternation of the color contrast attributes of the adjacent stimulus areas. Dynamic Optotype provides multiple improvements compared to detection of an angular separation of static points used with historical VA testing devices: application to various aspects of visual function, higher precision, higher reproducibility over both time and from subject to subject, higher test time efficiency, less confusion of endpoints, and insensitivity to cultural and literacy biases.

### BRIEF DESCRIPTION OF THE SEVERAL VIEWS OF THE DIAGRAMS

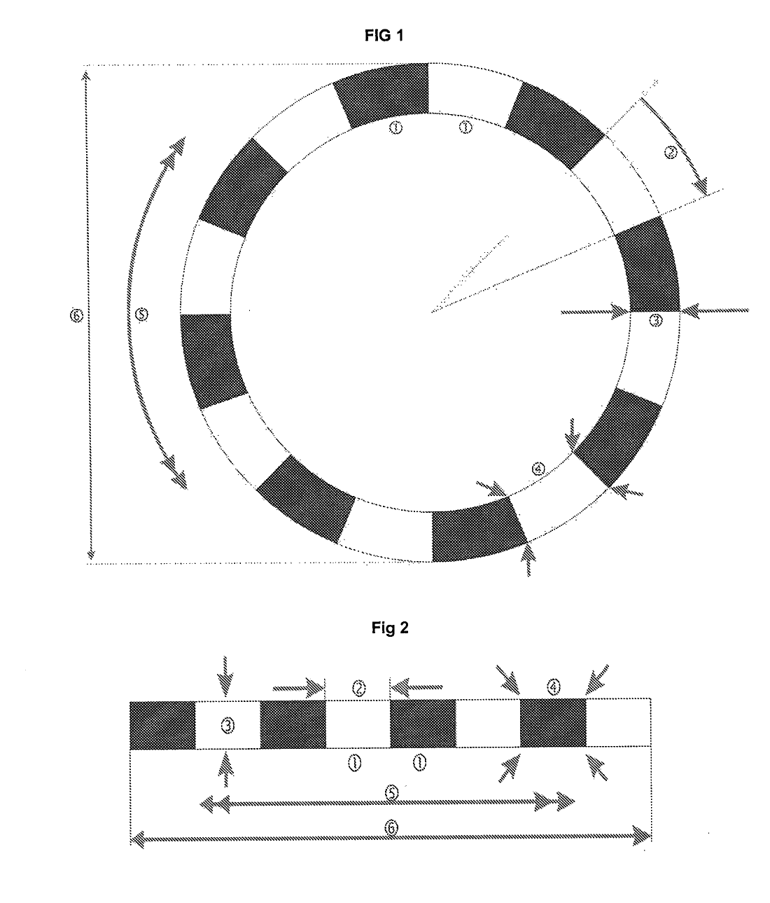

FIG. 1: Display of curvilinear variant of Dynamic Optotype components

Item 1—alternating segment colors

Item 2—segment visual angle

Item 3—segment visual arc-width scaled as % of total Dynamic Optotype diameter

Item 4—segment visual arc-area

Item 5—visual angular velocity or strobic contrast response

Item 6—Dynamic Optotype total visual angle

FIG. 2: Display of linear variant of Dynamic Optotype components

Item 1—alternating segment colors

Item 2—segment visual angle

Item 3—segment visual arc-width as % of total Dynamic Optotype diameter

Item 4—segment visual arc-area

Item 5—visual angular velocity or strobic contrast response

Item 6—Dynamic Optotype total visual angle

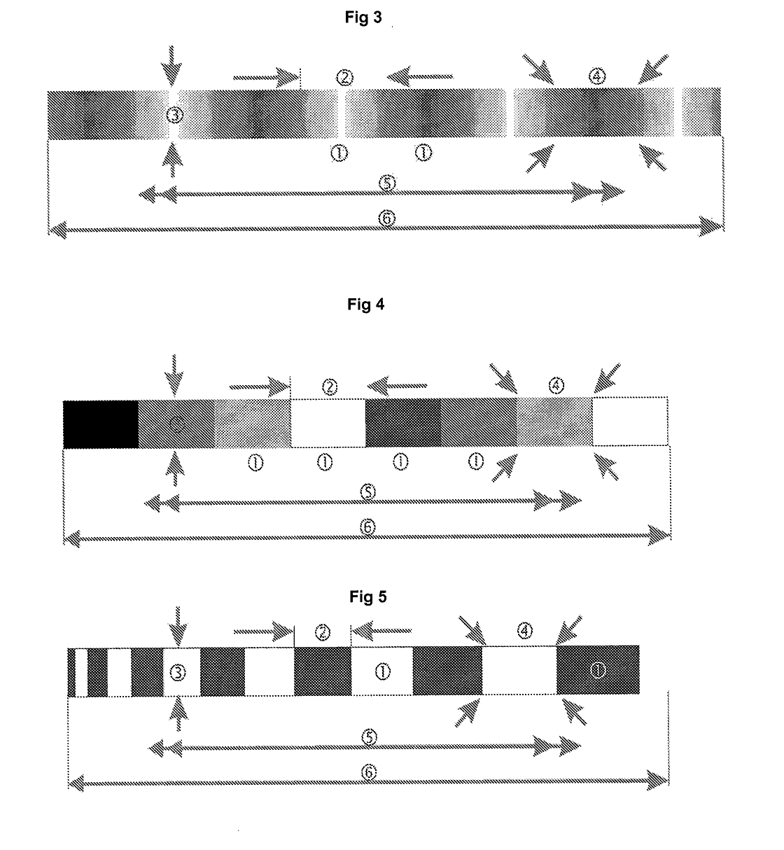

FIG. 3: Display of linear gradated variant of Dynamic Optotype components

Item 1—alternating segment colors

Item 2—segment visual angle

Item 3—segment visual arc-width as % of total Dynamic Optotype diameter

Item 4—segment visual arc-area

Item 5—visual angular velocity or strobic contrast response

Item 6—Dynamic Optotype total visual angle

FIG. 4: Display of linear multiple color/contrast variant of Dynamic Optotype components

Item 1—alternating segment colors

Item 2—segment visual angle

Item 3—segment visual arc-width as % of total Dynamic Optotype diameter

Item 4—segment visual arc-area

Item 5—visual angular velocity or strobic contrast response

Item 6—Dynamic Optotype total visual angle

FIG. 5: Display of a plurality linear variant of Dynamic Optotype components

Item 1—alternating segment colors

Item 2—segment visual angle

Item 3—segment visual arc-width as % of total Dynamic Optotype diameter

Item 4—accelerating or decelerating segment visual arc-areas

Item 5—accelerating or decelerating visual angular velocity or strobic contrast response

Item 6—Dynamic Optotype total visual angle

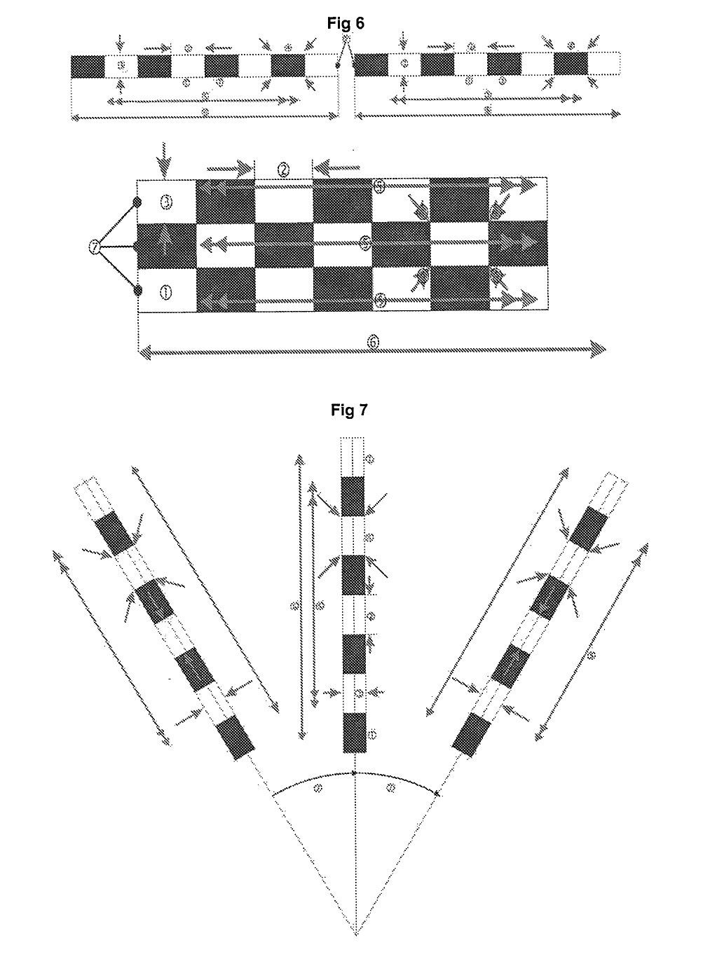

FIG. 6: Display of a plurality linear variant of Dynamic Optotype components

Item 1—alternating segment colors

Item 2—segment visual angle

Item 3—segment visual arc-width as % of total Dynamic Optotype diameter

Item 4—segment visual arc-area

Item 5—visual angular velocity or strobic contrast response

Item 6—Dynamic Optotype total visual angle

Item 7—plurality of Dynamic Optotypes with example positioning

FIG. 7: Display of multiple linear inter-angled variant of Dynamic Optotype components

Item 1—alternating segment colors

Item 2—segment visual angle

Item 3—segment visual arc-width as % of total Dynamic Optotype diameter

Item 4—segment visual arc-area

Item 5—visual angular velocity or strobic contrast response

Item 6—Dynamic Optotype total visual angle

Item 7—inter-optotype angle

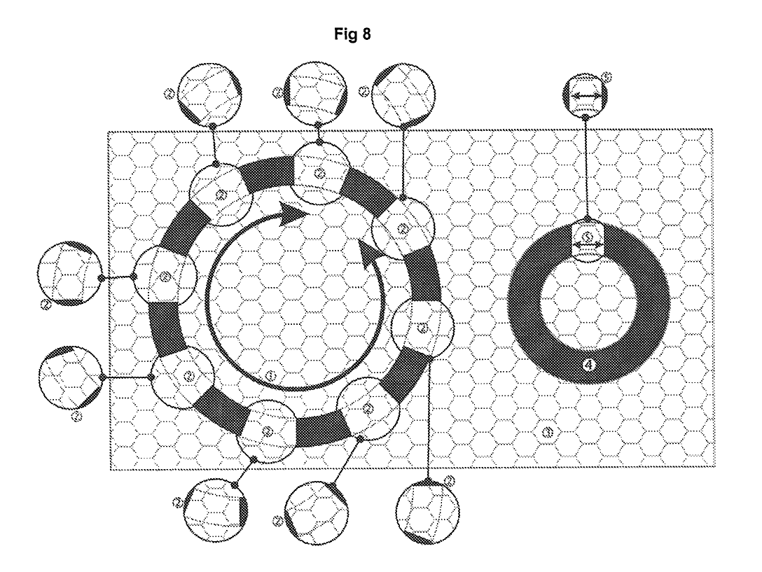

FIG. 8: Display of a plurality of moving segment areas from Dynamic Optotype imaged on the retina and the resultant increase in total discrete, moving, stimulated areas by summation of the individual areas

Item 1—visual angular velocity or strobic contrast response

Item 2—a moving segment visual arc-area dynamically stimulating retina cells with motion

Item 3—retinal cells

Item 4—an example of a static historical optotype

Item 5—a static minimum angle of resolution of a historical optotype

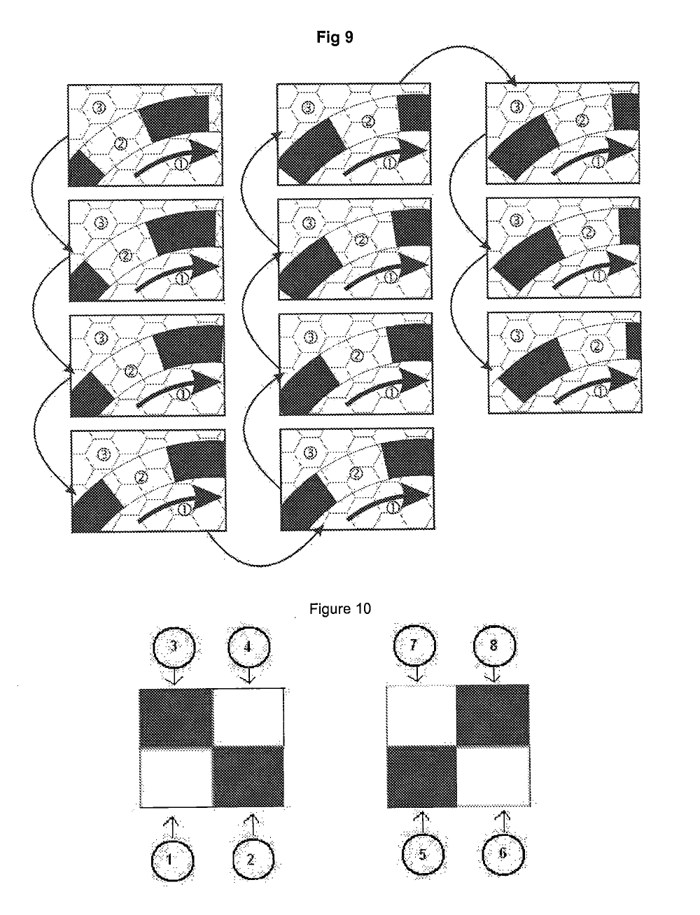

FIG. 9: Illustration of change in stimulus from movement over time of imaged Dynamic Optotype segments on retinal cells

Item 1—visual angular velocity or strobic contrast response

Item 2—a moving segment visual arc-area dynamically stimulating retina cells

Item 3—retinal cells

FIG. 10: Display of stationary contrasting strobic Dynamic Optotype components

Item 1—first strobic segment colors and visual stimulus area

Item 2—second strobic segment colors and visual stimulus area

Item 3—first strobic segment colors and visual stimulus area

Item 4—second strobic segment colors and visual stimulus area

Item 5—alternating first strobic segment colors and visual stimulus area

Item 6—alternating second strobic segment colors and visual stimulus area

Item 7—alternating first strobic segment colors and visual stimulus area

Item 8—alternating second strobic segment colors and visual stimulus area

1. We claim that a test using one or more linear or curvilinear, periodic, strobic contrast response or moving (non-static) geometric images (“Dynamic Optotype”) each composed of multiple, alternating, geometric (or amorphous), calibrated visual arc-areas. The Dynamic Optotypes may have either sharp borders between adjacent segment areas or gradated transitions, such as a sinusoidal or Gaussian blur. The Dynamic Optotype's visual arc-areas are varied by one or more features to illicit details specific to the studied visual function such as area size, strobic contrast response or velocity of motion (or no motion as a reference or comparative to the moving optotype), acceleration, direction or directions of motion, relative contrast of adjacent areas, colors or relative colors of the adjacent areas, or periodic frequency of the adjacent areas. Dynamic Optotype variables are controlled to elicit a subject's observation of either movement or no-movement to:
a. Assess visual acuity;
b. Assess precision of visual detection of velocity or relative velocities of a plurality of objects;
c. Assess refractive characteristics of the eye such as myopia, hyperopia, accommodation, or astigmatism amount or axis;
d. Assess visual contrast sensitivity;
e. Assess depth perception;
f. Assess color vision deficiencies;
g. Assess reduced sensitivity to color ranges;
h. Assess reduced sensitivity to color ranges such that relative deficiencies or strengths can be attenuated or augmented with color filters;
i. Assess or provide feedback on distance between the observer and the dynamic optotype including dynamic variation in optotype characteristics in conjunction with velocity of the observer or transporter of the dynamic optotype;
j. Assess nominal threshold detection of distances between observer and dynamic optotype; or,
k. Assess preferential viewing.
